(19) United States
(12) Patent Application Publication
(10) Pub. No.: US 2024/0226499 A1
(43) Pub. Date: Jul. 11, 2024

(54) SMART DEEP BREATHING MEDITATION DEVICE

(71) Applicant: Kwang Hyun JANG, Jeollanam-do (KR)

(72) Inventor: Kwang Hyun JANG, Jeollanam-do (KR)

(21) Appl. No.: 18/613,062

(22) Filed: Mar. 21, 2024

Related U.S. Application Data
(63) Continuation of application No. PCT/KR2023/013330, filed on Sep. 6, 2023.

(30) Foreign Application Priority Data
Sep. 21, 2022 (KR) 10-2022-0119056

(51) Int. Cl. A61M 21/02; A61M 21/00

(52) U.S. Cl. CPC A61M 21/02; A61M 2021/0022; A61M 2021/0088; A61M 2205/3592; A61M 2205/505; A61M 2205/584; A61M 2205/8243; A61M 2209/088; A61M 2210/083; A61M 2230/06; A61M 2230/42

(57) ABSTRACT

A smart deep breathing meditation device according to an embodiment of the present invention includes a housing body having an accommodation structure for accommodating a smartwatch and a contact surface, which is in contact with and attached to a part of a user's body and formed on one side surface thereof, a wire mounting portion formed on an upper surface or each of both side surfaces of the housing body and having a structure on which a necklace wire worn around the user's neck is mounted, a vibration providing unit which is mounted on the contact surface of the housing body, is operated by a control signal of a control unit, and provides an inspiration induction signal and an expiration induction signal by applying vibrations to skin of the part of the user's body, and the control unit which is mounted inside the housing body, wirelessly interworks with the smartwatch and a smartphone, receives heart rate data and breathing data of the user detected from the smartwatch in real time to control an operation of the vibration providing unit according to a preset control value.

### CROSS-REFERENCE TO RELATED APPLICATION

This application is a continuation application of the International PCT application serial no. PCT/KR2023/013330, filed on Sep. 6, 2023, which claims the priority benefit of Korea application no. 10-2022-0119056, filed on. The entirety of each of the above-mentioned patent applications is hereby incorporated by reference herein and made a part of this specification.

### TECHNICAL FIELD

The present invention relates to a smart deep breathing meditation device, and more specifically, to a smart deep breathing meditation device which includes components that induce a user in a breathing state to effectively enter a meditation state using a smartwatch.

### BACKGROUND ART

The number of people practicing meditation to stabilize mind and improve health is gradually increasing. However, proper self-practice meditation is very difficult, and long-term training is required to master the meditation.

Therefore, meditation is practiced together with an instructor in a training center, is practiced by watching video materials and following actions, or is practiced using audio materials. When content on a screen is continuously watched or a melody or sound with a preset certain pace is listened to follow breathing, due to the inconveniences and difficulties such as having to pay constant attention and frequently operate devices, there are many cases in which meditation practice is abandoned midway.

Therefore, there is a need for a tool in which even people without knowledge of meditation can feel that their mind is at ease through deep breathing and can observe their emotions and look into their mind through deep breathing.

### DISCLOSURE

### Technical Problem

The present invention is directed to providing a smart deep breathing meditation device in which even people without knowledge of meditation may feel that their mind is at ease through deep breathing and may observe their emotions and look into their mind through deep breathing and which may be easily used by being worn around a user's neck and may detect a user's physical condition and breathing state in real time and then may induce a change in breathing state based on the detected information such that a user effectively enters a meditation state.

### Technical Solution

One aspect of the present invention provides a smart deep breathing meditation device including a housing body having an accommodation structure for accommodating a smartwatch and a contact surface, which is in contact with and attached to a part of a user's body and formed on one side surface thereof, a wire mounting portion formed on an upper surface or each of both side surfaces of the housing body and having a structure on which a necklace wire worn around the user's neck is mounted, a vibration providing unit which is mounted on the contact surface of the housing body, is operated by a control signal of a control unit, and provides an inspiration induction signal and an expiration induction signal by applying vibrations to skin of the part of the user's body, and the control unit which is mounted inside the housing body, wirelessly interworks with the smartwatch and a smartphone, receives heart rate data and breathing data of the user detected from the smartwatch in real time to control an operation of the vibration providing unit according to a preset control value.

The vibration providing unit may be provided as a plurality of vibration providing units which are mounted to be spaced apart from each other at a predetermined angle based on a center of the contact surface of the housing body to form a circular space with a certain diameter at a central portion thereof, and each of which independently operates and provides the inspiration induction signal and the expiration induction signal by combining a vibration generation position, a vibration generation duration, the number of vibration generation, and a vibration generation period.

The housing body may include a power storage portion mounted inside the housing body and having a wirelessly chargeable structure, a waterproof structure forming portion formed on an inner surface of the housing body and formed to prevent liquid foreign materials from penetrating from the outside into an interior, and a heat exchange portion which is mounted inside the waterproof structure forming portion, is continuously mounted forward along an edge from the contact surface of the housing body, and is operated by a control signal of the control unit to absorb heat from the skin of the part of the user's body and then discharge the absorbed heat forward of the housing body.

When the heart rate data and the breathing date of the user enter an excited state, the control unit may operate the heat exchange portion according to a preset control value to apply cold air to the skin of the part of the user's body and simultaneously may operate the vibration providing unit to warn the user that deep breathing and meditation are necessary.

The housing body may further include a light-emitting portion continuously mounted along an edge of the housing body and operated by a control signal of the control unit to emit light with a preset color and pattern to the outside.

When the heart rate data and the breathing data of the user enter the excited state, the control unit may operate the light-emitting portion according to the preset control value to warn the user that deep breathing and meditation are necessary, and the control unit may combines a color of light, an emission duration, the number of emission, and an emission period to provide the inspiration induction signal and the expiration induction signal together with the vibration providing unit.

The smart deep breathing meditation device may include an upper dantian induction portion which has a structure that is attached to and detached from the user's upper dantium and in which a vibration providing unit is mounted at a portion in contact with the user's skin, a light-emitting portion is mounted at an edge, and a heat exchange portion is continuously mounted at the edge from the portion in contact with the user's skin, and a lower dantian induction portion which has a structure that is attached to and detached from the user's lower dantium and in which a vibration providing unit is mounted at a portion in contact with the user's skin, a light-emitting portion is mounted at an edge, and a heat exchange portion is continuously mounted at the edge from the portion in contact with the user's skin.

The light-emitting portion may be operated by a control signal of a control unit to emit light with a preset color and pattern to the outside, and the heat exchange portion may be operated by a control signal of the control unit to absorb heat from skin of a part of a user's body and then discharge the absorbed heat forward of a housing body.

The control unit of the smart deep breathing meditation device may operate after storing a control method of operating the smart deep breathing meditation device therein in a form of a program, and the control method of operating the smart deep breathing meditation device may include a user physical information input operation of wirelessly interworking with the smartwatch and the smartphone in a state of being worn around the user's neck or wrist and then wirelessly interworking with the smartwatch and the smartphone of the user to input physical information of the user using an application installed on the smartphone, a user physical information detection operation of detecting physical information of the user such as a heart rate, a body temperature, and breathing in real time to store the detected physical information in the control unit, an emergency situation detection operation of determining, by the control unit, based on the physical information data of the user, whether a physical condition of the user is a condition in which meditation is necessary and furthermore whether the physical condition is in an emergency situation, an emergency rescue request operation of, when the physical condition of the user is in the emergency situation, transmitting the physical condition of the user to an emergency rescue center in real time, an input signal detection operation of, when the physical condition of the user is the condition in which meditation is necessary, determining two cases of condition A and condition B, an input value reflection meditation induction performing operation of, under condition A, when the physical condition of the user enters a standard range preset by the user, performing meditation immediately without asking the user whether to start meditation, a physical condition reflection meditation induction performing operation of, under condition B, when the physical condition enters another standard range set by the user, asking the user whether to start meditation, and when an operation button input or a touch button input is performed, performing meditation according to a preset program, and a meditation end determination operation of, when a time to complete a meditation process according to the preset program is imminent, asking the user whether to end the meditation and whether to continue the meditation.

### Advantageous Effects

As described above, a smart deep breathing meditation device according to the present embodiment includes a housing body having a specific structure, a wire mounting portion, a vibration providing unit, and a control unit. Thus, there can be provided the smart deep breathing meditation device in which even people without knowledge of meditation can feel that their mind is at ease through deep breathing and observe their emotions and look into their mind through deep breathing and which can be easily used by being worn around a user's neck, detect a user's physical condition and breathing state in real time, and then induce a change in breathing state based on detected information such that a user effectively enters a meditation state.

In addition, according to a smart deep breathing meditation device according to the present embodiment, there can be provided the smart deep breathing meditation device which is capable of solving problems of the related art in which, since meditation requiring concentration is a self-practice performed at a set certain time and place, there are many difficulties in practicing the meditation which allows a user to freely and conveniently practice meditation irrespective of time and place.

Furthermore, a smart deep breathing meditation device according to the present can induce formal meditation and informal mediation to be easily practiced, wherein the formal meditation is performed at a set certain time and place as a self-practice for controlling his or her mind by concentrating his or her mind through breathing and the informal meditation is performed in a free manner. The smart deep breathing meditation device can be used as a formal meditation device that allows a user to meditate at a certain time and place, can also be used as an informal meditation device that allows a user to freely meditate at a convenient time, and can also be used as a portable meditation device that is carried to allow a user to meditate anytime and anywhere at his or her intended time.

In addition, a smart deep breathing meditation device according to the present invention is a necklace-type (middle dantian) meditation device that induces a user to concentrate his or her mind through deep breathing according to a vibration rhythm when the earth vibrates. The smart deep breathing meditation device allows a user to meditate by concentrating his or her mind through a sensation of vibration vibrating in the chest.

In addition, according to a smart deep breathing meditation device of the present invention, a user can meditate at any time irrespective of a method, procedure, a posture, or a place of meditation by setting a vibration notification at a desired time and pattern.

### DESCRIPTION OF DRAWINGS

FIG. 1 is a diagram illustrating a configuration of a meditation inducing device according to the related art.

FIG. 2 a perspective view illustrating a state in which a smart deep breathing meditation device according to one embodiment of the present invention is separated from a smartwatch.

FIG. 3 is a front view illustrating the smart deep breathing meditation device shown in FIG. 2.

FIG. 4 is a front view illustrating a vibration providing unit of the smart deep breathing meditation device shown in FIG. 3.

FIG. 5 is a front view illustrating a heat exchange portion of the smart deep breathing meditation device shown in FIG. 3.

FIG. 6 is a block diagram illustrating the smart deep breathing meditation device according to one embodiment of the present invention.

FIG. 7 is a schematic diagram illustrating a state in which the smart deep breathing meditation device according to one embodiment of the present invention is worn on a chest as a necklace.

FIG. 8 is a control flowchart illustrating a method of operating a smart deep breathing meditation device according to one embodiment of the present invention.

FIG. 9 is a perspective view illustrating a state in which a strap is mounted on a wire mounting portion of a smart deep breathing meditation device according to another embodiment of the present invention to use the smart deep breathing meditation device as a smartwatch worn on a user's wrist.

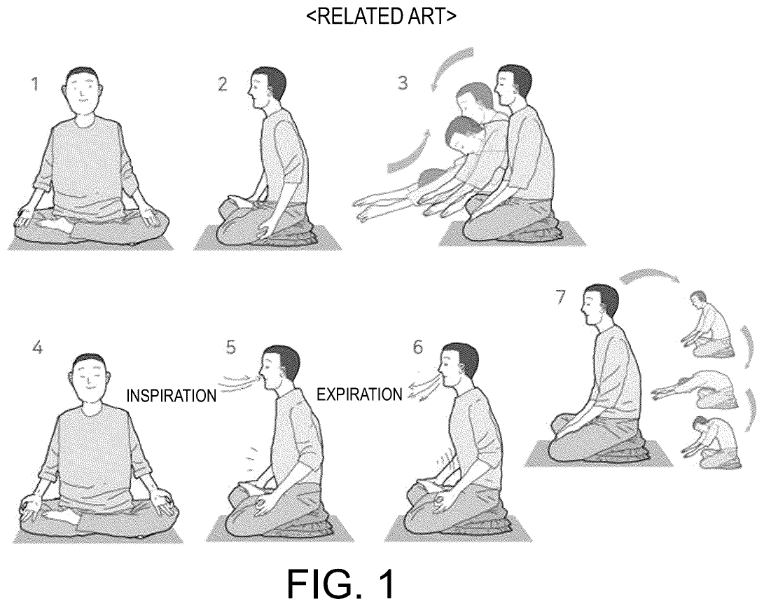

### BEST MODES OF THE INVENTION

Hereinafter, exemplary embodiments of the present invention will be described with reference to the accompanying drawings. Prior to this, terms and words used in the present specification and claims have to be interpreted by not the limited meaning of the typical or dictionary definition, but the meaning and concept corresponding to the technical idea of the present invention.

Throughout the present application, a description of a first member being placed “on” a second member includes not only a case of the first member being in contact with the second member but a case of a third member being present between the two members. Throughout the present specification, unless explicitly described to the contrary, the word “comprise” and variations such as “comprises” or “comprising” will be understood to imply the inclusion of stated elements but not the exclusion of any other elements.

Breathing is respiration in which a breath is inhaled and exhaled unconsciously by the autonomic nerves of the brain.

Deep breathing is respiration in which a breath is intentionally inhaled and exhaled in his or her spirit.

In addition, meditation is the act of looking into his or her mind through breathing.

The meditation is for concentrating mind to become aware of his or her thoughts and emotions.

A smart deep breathing meditation device is a meditation device that allows a user to concentrate mind through deep breathing.

The smart deep breathing meditation device allows the user to improve thoughts, words, and actions.

There are various ways to meditate, and there are formal meditation and informal meditation.

The formal meditation is meditation that is performed at a certain time and place.

The informal meditation is meditation that is freely performed anytime and anywhere.

Breathing is mainly divided into lower abdomen breathing and thoracic breathing.

The lower abdomen breathing is suitable for the formal meditation performed in a stable posture.

The thoracic breathing is suitable for the informal meditation freely performed.

Anyone may become aware of their state of mind and emotions in their spirit.

Therefore, when the smart deep breathing meditation device vibrates,
since meditation for becoming aware of his or her state of mind and emotions is repeated every day,
his or her thoughts, words, and actions may be correctly improved.

The smart deep breathing meditation device is a portable meditation device that induces meditation by frequently setting inspiration for a few seconds and expiration for a few seconds
every few minutes to several hours during lower abdomen breathing and thoracic breathing
according to his or her intention.

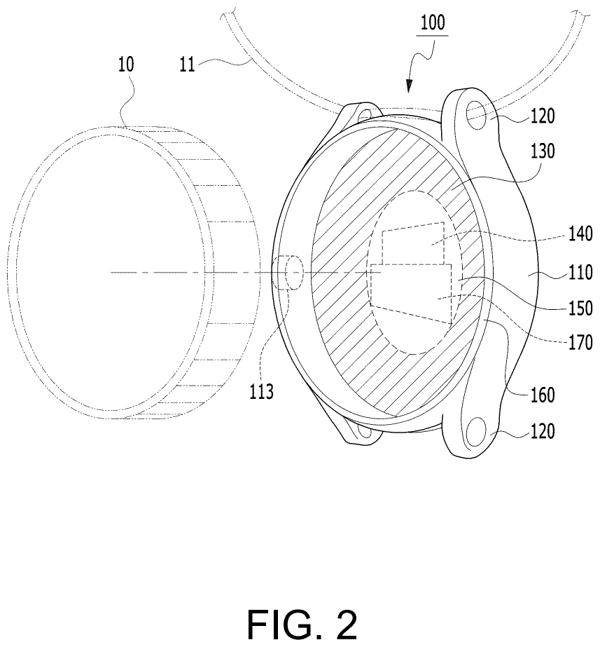

FIG. 2 a perspective view illustrating a state in which a smart deep breathing meditation device according to one embodiment of the present invention is separated from a smartwatch.

Referring to FIG. 2, a smart deep breathing meditation device 100 according to the present embodiment includes a housing body 110 having a specific structure, a wire mounting portion 120, a vibration providing unit 130, and a control unit 140. Thus, there may be provided the smart deep breathing meditation device in which even people without knowledge of meditation may feel that their mind is at ease through deep breathing and may observe their emotions and look into their mind through deep breathing and which may be easily used by being worn around a user's neck and may detect the user's physical condition and breathing state in real time and then may induce a change in breathing state based on the detected information such that the user effectively enters a meditation state.

Deep breathing is respiration in which a breath is inhaled and exhaled deeply. Humans breathe unconsciously through the autonomic nerve of the brain, but deep breathing is respiration in which a breath is inhaled and exhaled intentionally. Meditation is for looking into his or her mind by concentrating his or her mind and is for becoming aware of his or her mind and emotions by giving his or her mind. In addition, deep breathing meditation is for concentrating consciousness, breathing deeply, and stabilizing mind to adjust his or her thoughts and emotions.

There are various ways to meditate, and there are formal meditation and informal meditation. The formal meditation is meditation performed at a set certain time every day or week, and the informal meditation is meditation performed in a comfortable and free manner anytime and anywhere.

The smart deep breathing meditation device according to the present embodiment is used as a formal meditation device that allows the user to meditate a certain time interval and is also used as an informal meditation device that allows the user to frequently meditate at a convenient time, is a device that allows the user to meditate by looking into his or her mind and emotions again and again through deep breathing, is a training device that allows the user to adjust and control his or her thoughts and emotions with his or her intention, and is a portable meditation device that may be conveniently used by anyone.

The smart deep breathing meditation device according to the present embodiment allows the user to meditate immediately through deep breathing in any situation irrespective of a method and procedure of meditation during work at a job, in a noisy crowed busy place, or on a bench in a leisurely park.

In this way, when deep breathing which is beneficial for body and mind is frequently taken tens to hundreds of times a day through meditation, it will be healthy self-discipline of taking care of his or her body and introspecting mind.

People may look into their mind through frequent deep breathing meditation, may offer prayers of grace and gratitude by practicing deep breathing meditation from time to time, and may cultivate a heart of forgiveness and mercy by practicing deep breathing meditation from time to time.

Hereinafter, each component constituting the smart deep breathing meditation device 100 according to the present embodiment will be described in detail with reference to the accompanying drawings.

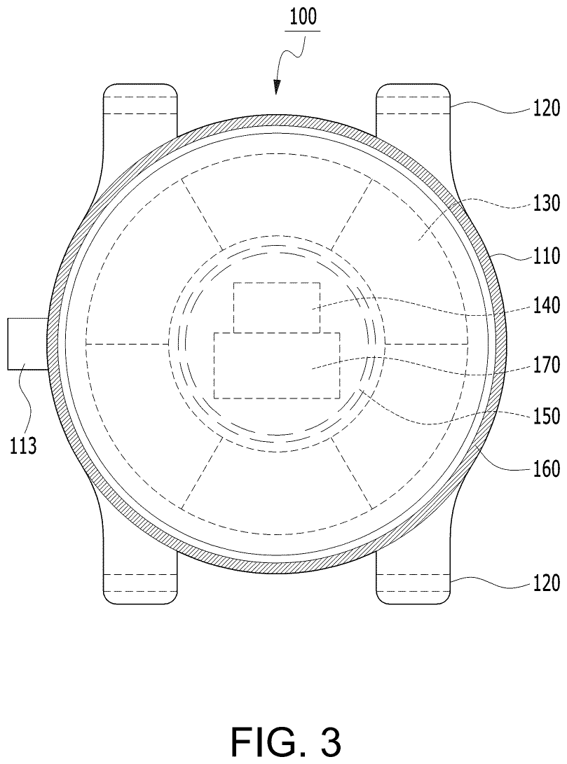

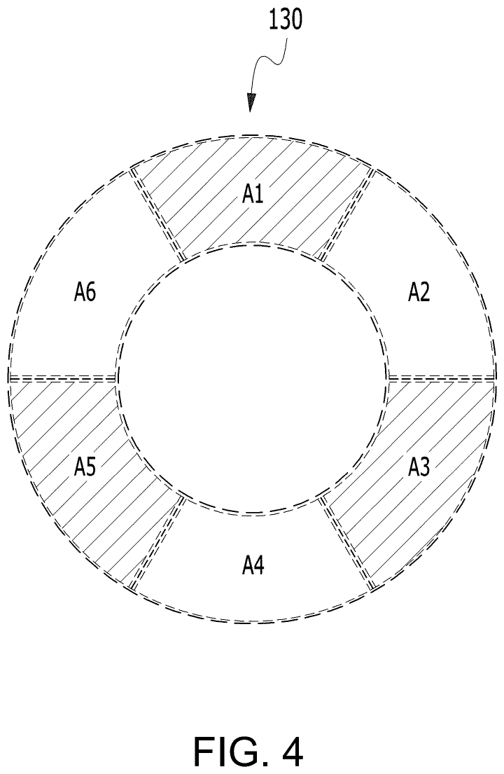

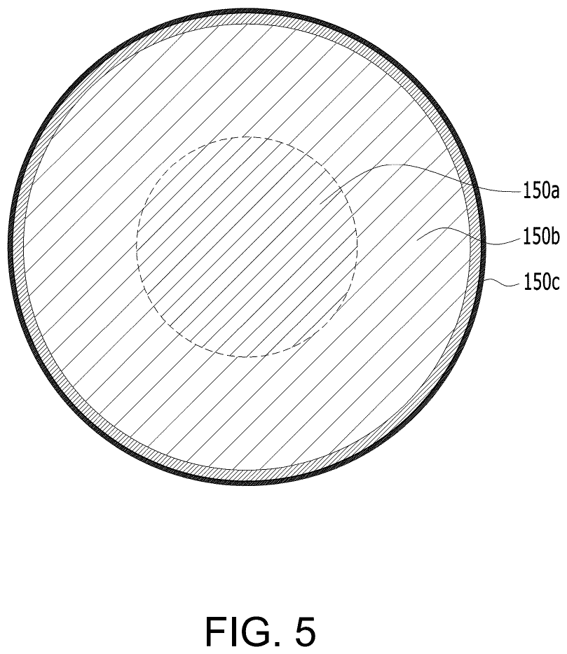

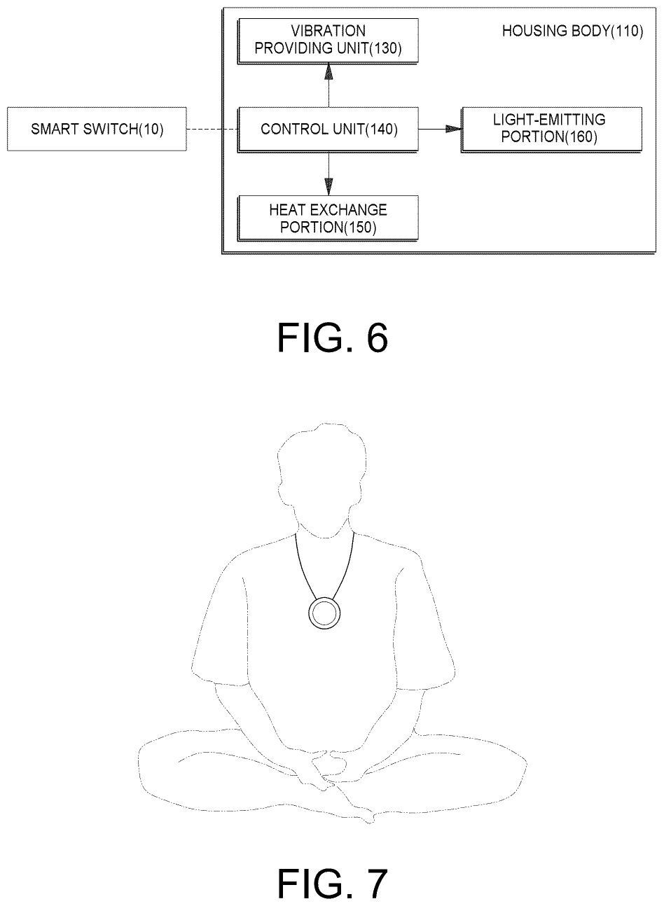

FIG. 3 is a front view illustrating the smart deep breathing meditation device shown in FIG. 2. FIG. 4 is a front view illustrating a vibration providing unit of the smart deep breathing meditation device shown in FIG. 3. FIG. 5 is a front view illustrating a heat exchange portion of the smart deep breathing meditation device shown in FIG. 3. In addition, FIG. 6 is a block diagram illustrating the smart deep breathing meditation device according to one embodiment of the present invention. FIG. 7 is a schematic diagram illustrating a state in which the smart deep breathing meditation device according to one embodiment of the present invention is worn on a chest as a necklace.

Referring to the drawings, the housing body 110 according to the present embodiment has an accommodation structure for accommodating a smartwatch 10 and is formed in a structure in which a contact surface is formed on one side surface thereof that may be attached in contact with a part of a user's body.

Specifically, as shown in FIGS. 2 and 3, the housing body 110 may be a component including a power storage portion 170 with a specific structure and a waterproof structure forming portion. The power storage portion 170 mounted inside the housing body 110 preferably has a structure that may be wirelessly charged. In addition, the waterproof structure forming portion is preferably mounted on an inner surface of the housing body 110 to prevent liquid foreign materials from penetrating from the outside into the interior.

A wire mounting portion 120 according to the present embodiment is formed on an upper surface or each of both side surfaces of the housing body 110 and has a structure in which a necklace wire that may be worn around the user's neck is mounted.

In some cases, a strap with a structure that wraps a wrist may be attached to the wire mounting portion 120 to use the smart deep breathing meditation device as a smartwatch.

The vibration providing unit 130 according to the present embodiment may be a component mounted on the contact surface of the housing body 110 and may be operated by a control signal of the control unit 140 to apply vibrations to the skin of a part of the user's body and provide an inspiration induction signal and an expiration induction signal.

Specifically, as shown in FIGS. 3 and 4, a plurality of vibration providing units 130 may be installed around the center of the contact surface of the housing body 110 to form a circular space with a certain diameter at a central portion thereof. In this case, each of the plurality of vibration providing units 130 independently operates. In this case, the vibration providing unit 130 according to the present embodiment may provide an inspiration induction signal and an expiration induction signal by combining a vibration generation position, a vibration generation duration, the number of vibration generation, and a vibration generation period of the plurality of vibration providing units 130.

The control unit 140 according to the present embodiment may be mounted inside the housing body 110, may wirelessly interwork with a smartwatch and a smartphone, and may receive heart rate data and breathing data of the user detected from the smartwatch in real time to control the operation of the vibration providing unit 130 according to a preset control value.

In one embodiment, the user may select and operate a breathing method consisting of deep breathing or abdominal breathing. In this case, the breathing method may be performed by performing functions of notifying the user of a breathing attempt through vibrations, providing vibration notification at an interval of 1 minute to 10 minutes during formal meditation (sit-down meditation), and providing vibration notification at an interval of 10 minutes to 180 minutes during informal meditation (meditation in daily life anytime and anywhere).

As a vibration method, breathing may be set to be short or long, and vibrations for inspiration may be made to sound like “beep beep beep,” thereby inducing the user to quickly realize vibrations. Vibrations for expiration may induce a breath to be exhaled by making vibrations continue for a long time. During 4-second vibration deep breathing, vibrations for inspiration may be set as “beep” for 2 seconds, during 6-second vibration deep breathing, vibrations for inspiration may be set as “beep beep beep” for 3 seconds, and vibrations for expiration may be set as “beep” for 3 seconds, during 8-second vibration deep breathing, vibrations for inspiration may be set as “beep beep beep beep” for 4 seconds, and vibrations for expiration may be set as “beep” for 4 seconds, during 10-second vibration deep breathing, vibrations for inspiration may be set as “beep beep beep beep beep” for 5 seconds, and vibrations for expiration may be set as “beep” for 5 seconds, and during 12-second vibration deep breathing, vibrations for inspiration may be set as “beep beep beep beep beep beep” for 6 seconds, and vibrations for expiration may be set as “beep” for 6 seconds. The intensity of vibration may be adjusted (may be adjusted by being subdivided from level 1 to level 5), and a frequency (Hz) may be adjusted.

An abdominal breathing function may be set to 10-second abdominal breathing, and a breath is inhaled for 3 seconds of “beep,” is held for 3 seconds, and then is exhaled for 4 seconds.

In some cases, the smart deep breathing meditation device 100 according to the present embodiment may be a component further including a heat exchange portion 150 with a specific structure.

Specifically, as shown in FIGS. 3 and 5, the heat exchange portion 150 may be a component mounted inside the waterproof structure forming portion, may be continuously mounted forward along an edge from the contact surface of the housing body 110, and may be operated by a control signal of the control unit 140 to absorb heat from the skin of a part of a user's body and then discharge the absorbed heat forward of the housing body 110.

In this case, when heart rate data and breathing date of a user enter an excited state, the control unit 140 according to the present embodiment may operate the heat exchange portion 150 according to a preset control value to apply cold air to the skin of a part of a user's body and simultaneously may operate the vibration providing unit 130 to warn a user that deep breathing and meditation are necessary.

The smart deep breathing meditation device 100 according to the present embodiment may be a component further including a light-emitting portion 160 with a specific structure.

Specifically, as shown in FIGS. 2 and 3, the light-emitting portion 160 is a component continuously mounted along an edge of the housing body 110 and is operated by a control signal of the control unit 140 to emit light with a preset color and pattern to the outside.

In this case, when heart rate data and breathing data of a user enter an excited state, the control unit 140 according to the present embodiment may operate the light-emitting portion 160 according to a preset control value to warn a user that deep breathing and meditation are necessary. In addition, the control unit 140 preferably combines a color of light, an emission duration, the number of emission, and an emission period to provide an inspiration induction signal and an expiration induction signal together with the vibration providing unit 130.

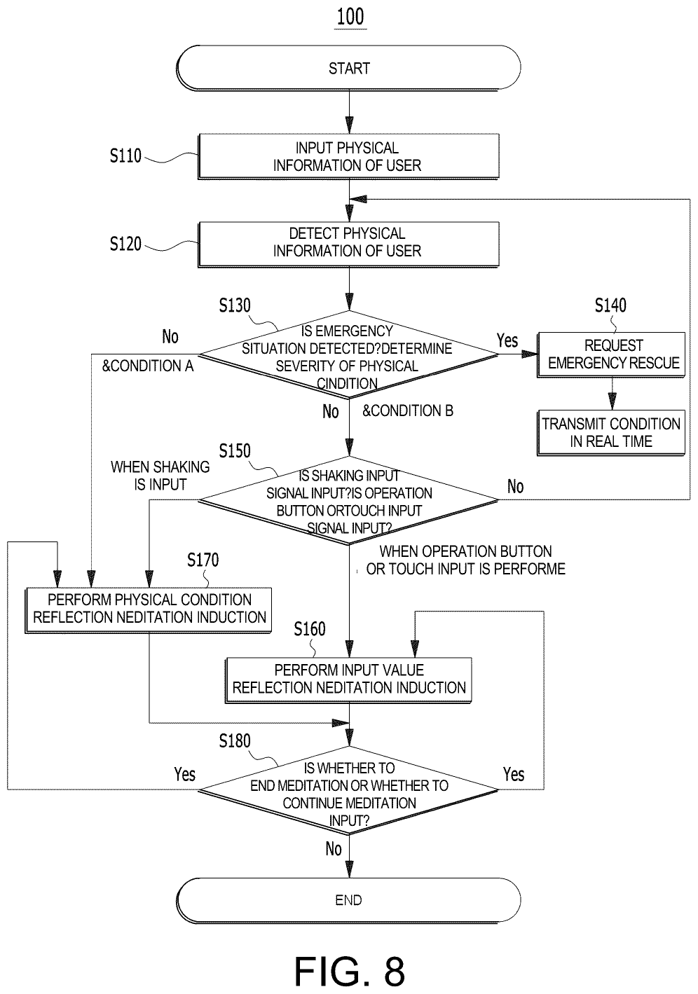

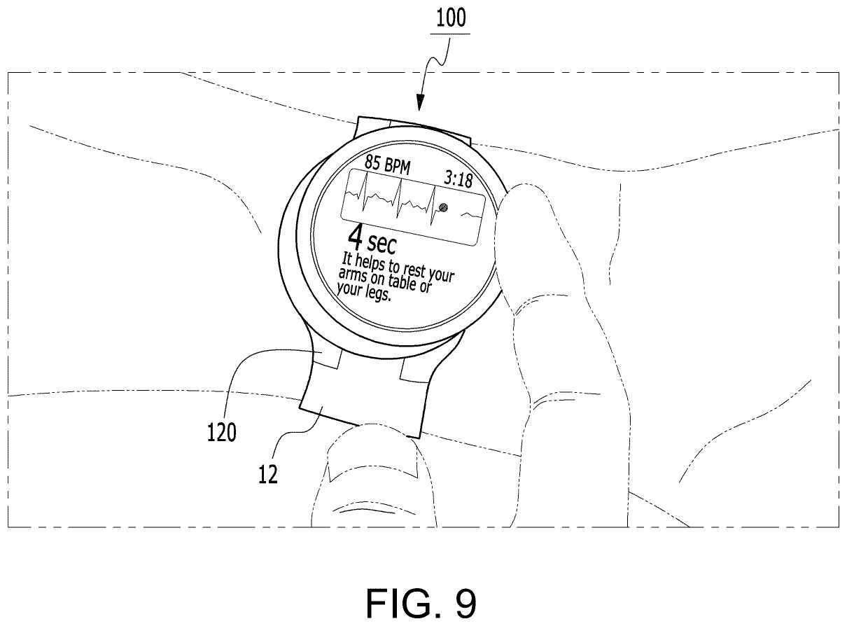

FIG. 8 is a control flowchart illustrating a method of operating a smart deep breathing meditation device according to one embodiment of the present invention. FIG. 9 is a perspective view illustrating a state in which a strap is mounted on a wire mounting portion of a smart deep breathing meditation device according to another embodiment of the present invention to use the smart deep breathing meditation device as a smartwatch worn on a user's wrist.

Referring to the drawings, as shown in FIG. 9, a strap with a structure wrapping a user' wrist may be selected and applied to a wire mounting portion 120 to use a smart deep breathing meditation device 100 according to the present embodiment as a smartwatch.

A control unit 140 of the smart deep breathing meditation device 100 according to the present embodiment is preferably set to perform a specific process as shown in FIG. 9.

Specifically, the smart deep breathing meditation device 100 may wirelessly interwork with a smartwatch and a smartphone while worn around a user's neck or wrist. In this case, by wirelessly interworking with a smartwatch and a smartphone of a user, physical information of the user is input using an application installed on the smartphone (S110). Thereafter, the smart deep breathing meditation device 100 detects the physical information such as a heart rate, a body temperature, and breathing of the user in real time and stores the detected physical information in the control unit 140 (S120).

Based on physical information data acquired in real time, the control unit 140 determines whether a physical condition of the user is a condition in which meditation is necessary and furthermore whether the physical condition is in an emergency situation (S130). In this case, when the physical condition of the user is in an emergency situation, an emergency rescue is requested immediately, and the physical condition of the user is transmitted in real time to an emergency rescue center. On the other hand, when the physical condition of the user is a condition in which meditation is necessary, two cases of condition A and condition B are determined. Condition A is a condition of, when the physical condition of the user enters a standard range preset by the user, performing meditation immediately without asking the user whether to start meditation (S170). Condition B is a condition of, when the physical condition enters another standard range set by the user, asking the user whether to start meditation, and when an operation button input or a touch button is performed, performing meditation according to a preset program (S160).

When a time to complete a meditation process according to a preset program is imminent, the user is asked whether to end meditation and whether to continue meditation (S180).

Of course, a process of inducing meditation using the above-described preset program may be freely changed and set by the user.

For example, after vibrations are generated by matching a heart rate and a breathing rate of the user, changes may be induced to vibrations and patterns necessary for meditation to quickly induce deep breathing of the user to a comfortable state.

A smartwatch screen of the smart deep breathing meditation device may be used as a watch at ordinary times, and at each time set by himself or herself, a time for deep breathing meditation is notified with a vibration signal. In this case, a today's meditation text may be displayed on the smartwatch screen.

The “today's meditation” text will be described with “examples” as follows:

Take a deep breath and calm mind.

Take a deep breath and have a grateful heart.

Take a deep breath and have a forgiving heart.

Take a deep breath and have a kind heart.

Take a deep breath and speak properly.

Today's meditation text appears when a monitor is pressed and held (or another method).

When the monitor is pressed and held again, the text disappears.

A method of inputting a text on the monitor is performed by interworking with a smartphone or a personnel computer (PC) to input a text.

The smart deep breathing meditation device may be used as a formal meditation device that allows a user to meditate a certain time interval.

In addition, the smart deep breathing meditation device may also be used as an informal meditation device that allows a user to freely meditate at a convenient time.

The smart deep breathing meditation device may also be used as a training device that allows a user to improve his or her behavior through deep breathing.

A portable smart deep breathing meditation device is a convenient device that is carried to allow a user to meditate anytime and anywhere
irrespective of time and place.

The portable smart deep breathing meditation device allows a user to meditate immediately through deep breathing as set by himself or herself
in any situation irrespective of a method and procedure of meditation during work at a job, in a noisy crowed busy place, or on a bench in a leisurely park.

The effectiveness of deep breathing has already been scientifically proven through a modern medical science.

Through only few deep breathing, oxygen is smoothly supplied to the brain.

The deep breathing is effective in removing bad toxins and relieving unpleasant emotions.

Deep breathing assists in relieving stress, assists in stabilizing blood pressure, and relieves pain in the body.

Deep breathing has effects of stimulating blood circulation, excreting toxins in the body, and assisting in healthy sleep.

In addition, deep breathing releases endorphin that is a natural painkiller and lifts mood.

In this way, only few deep breathing assists in relieving stress.

Deep breathing assists in quickly settling mind in an unpleasant mental state.

Thus, deep breathing is effective in controlling bad emotions such as anger or fear.

Therefore, when deep breathing meditation for looking into his or her body and mind is frequently performed regularly every day in everyday life,
the deep breathing allows a user to control his or her body and mind and is good for health.

Meditation aims to control his or her mind and correct his or her thoughts, words, and actions.

The meditation aims to be applied to daily life to improve his or her life.

Therefore, anyone may focus their mind through a smart deep breathing meditation device.

Anyone may control their wrong desires and impulses and may improve the wrong desires and impulses properly.

For example, when anyone tries to quit smoking, tries to quit drinking, or tries to lose weight,
it will be difficult to succeed when continued desires and impulses may not be controlled.

On the other hand, when mind is frequently concentrated and stabilized through smart deep breathing meditation to look into and control his or her emotions,
a chance for success may be increased.

The smart deep breathing meditation device is a training device that allows a user to control his or her mind, control his or her emotions,
and improve his or her wrong personality and behavior.

Therefore, through such a smart meditation device,
anyone may control their mind and emotions by frequently practicing deep breathing meditation,
may frequently practice deep breathing meditation and offer prayers of love and gratitude, and
may frequently practice deep breathing meditation and may have courage and hope.

Meditation is for focusing on his or her inner side and
is not about competing or fighting with anyone.

The meditation is the act of looking his or her mind and creating his or her own happiness.

As people live, people acquire wisdom and knowledge through life experience and education.

However, when there is true knowledge that a human being must learn throughout his or her life,
it is the ability to control his or her body and mind through a self-development practice.

This ability may be said to be the most humane discipline.

Greater knowledge may be obtained.

This knowledge is a practice for self-improvement of noble value that should be sought and obtained no matter how much effort or price it takes.

It is a practice for self-development that has noble value that should be sought and achieved.

As described above, a smart deep breathing meditation device according to the present embodiment includes a housing body 110 having a specific structure, a wire mounting portion 120, a vibration providing unit 130, and a control unit 140. Thus, there may be provided the smart deep breathing meditation device in which even people without knowledge of meditation may feel that their mind is at ease through deep breathing and may observe their emotions and look into their mind through deep breathing and which may be easily used by being worn around a user's neck and may detect a user's physical condition and breathing state in real time and then may induce a change in breathing state based on detected information such that a user effectively enters a meditation state.

In the detailed description of the present invention, only special embodiments have been described. It is however to be understood that the present invention is not limited to the special embodiments, but should be construed as including all of changes, equivalents, and substitutes within the spirit and range of the present invention defined by the appended claims.

That is, the present invention is not limited to the above-described specific embodiments and descriptions. Various modifications may be made by anyone skilled in the art without departing from the gist of the present invention as claimed in the claims, and such modifications will fall within the scope of protection of the present invention.

### MODES OF THE INVENTION

Modes of the Invention have been described together in the above Best Modes of the Invention.

### INDUSTRIAL APPLICABILITY

The present invention relates to a smart deep breathing meditation device and has industrial applicability in that the smart deep breathing meditation device includes a housing body having a specific structure, a wire mounting portion, a vibration providing unit, and a control unit, thereby providing a smart deep breathing meditation device in which even people without knowledge of meditation may feel that their mind is at ease through deep breathing and may observe their emotions and look into their mind through deep breathing and which may be easily used by being worn around a user's neck and may detect the user's physical condition and breathing state in real time and then may induce a change in breathing state based on detected information such that the user effectively enters a meditation state.

1. A smart deep breathing meditation device comprising:
a housing body (110) having an accommodation structure for accommodating a smartwatch (10) and a contact surface, which is in contact with and attached to a part of a user's body and formed on one side surface thereof, and formed in a structure usable as a wristwatch using a cap made of silicone;
a protruding operation button (113) which is mounted to protrude to a certain height from a front surface or a side surface of the housing body (110), is touched on clothes in an unseen manner to control an operation of a control unit (140) through the user's pressing operation and allows a meditation time reservation time to be set;
a wire mounting portion (120) formed on an upper surface or each of both side surfaces of the housing body (110) and having a structure on which a necklace wire worn around the user's neck is mounted;
a vibration providing unit (130) which is mounted on the contact surface of the housing body (110), is operated by a control signal of the control unit (140), and provides an inspiration induction signal and an expiration induction signal by applying vibrations to skin of the part of the user's body; and
the control unit (140) which is mounted inside the housing body (110), wirelessly interworks with the smartwatch and a smartphone, receives heart rate data and breathing data of the user detected from the smartwatch in real time to control an operation of the vibration providing unit (130) according to a preset control value, and detects shaking, vibrations, and touch by the user to control the operation of the vibration providing unit (130).

2. The smart deep breathing meditation device of claim 1, wherein the vibration providing unit (130) is provided as a plurality of vibration providing units which are mounted to be spaced apart from each other at a predetermined angle based on a center of the contact surface of the housing body (110) to form a circular space with a certain diameter at a central portion thereof and each of which independently operates and provides the inspiration induction signal and the expiration induction signal by combining a vibration generation position, a vibration generation duration, the number of vibration generation, and a vibration generation period.

3. The smart deep breathing meditation device of claim 1, wherein the housing body (110) includes:
a power storage portion (170) mounted inside the housing body (110) and having a wirelessly chargeable structure;
a waterproof structure forming portion formed on an inner surface of the housing body (110) and formed to prevent liquid foreign materials from penetrating from an outside into an interior; and
a heat exchange portion (150) which is mounted inside the waterproof structure forming portion, is continuously mounted forward along an edge from the contact surface of the housing body (110), and is operated by a control signal of the control unit (140) to absorb heat from the skin of the part of the user's body and then discharge the absorbed heat forward of the housing body (110),
wherein, when the heart rate data and the breathing date of the user enter an excited state, the control unit (140) operates the heat exchange portion (150) according to a preset control value to apply cold air to the skin of the part of the user's body and simultaneously operates the vibration providing unit (130) to warn the user that deep breathing and meditation are necessary.

4. The smart deep breathing meditation device of claim 3, wherein the housing body (110) further includes a light-emitting portion (160) continuously mounted along an edge of the housing body (110) and operated by a control signal of the control unit (140) to emit light with a preset color and pattern to the outside,
when the heart rate data and the breathing data of the user enter the excited state, the control unit (140) operates the light-emitting portion (160) according to the preset control value to warn the user that deep breathing and meditation are necessary, and
the control unit (140) combines a color of light, an emission duration, the number of emission, and an emission period to provide the inspiration induction signal and the expiration induction signal together with the vibration providing unit (130).

5. The smart deep breathing meditation device of claim 1, wherein the control unit (140) of the smart deep breathing meditation device operates after storing a control method (S100) of operating the smart deep breathing meditation device (100) therein in a form of a program,
wherein the control method (S100) of operating the smart deep breathing meditation device includes:
a user physical information input operation (S100) of wirelessly interworking with the smartwatch and the smartphone in a state of being worn around the user's neck or wrist and then wirelessly interworking with the smartwatch and the smartphone of the user to input physical information of the user using an application installed on the smartphone;
a user physical information detection operation (S120) of detecting physical information of the user such as a heart rate, a body temperature, and breathing in real time to store the detected physical information in the control unit (140);
an emergency situation detection operation (S130) of determining, by the control unit (140), based on the physical information data of the user, whether a physical condition of the user is a condition in which meditation is necessary and furthermore whether the physical condition is in an emergency situation;
an emergency rescue request operation (S140) of, when the physical condition of the user is in the emergency situation, transmitting the physical condition of the user to an emergency rescue center in real time;
an input signal detection operation (S150) of, when the physical condition of the user is the condition in which meditation is necessary, determining two cases of condition A and condition B;
an input value reflection meditation induction performing operation (S160) of, under condition A, when the physical condition of the user enters a standard range preset by the user, performing meditation immediately without asking the user whether to start meditation;
a physical condition reflection meditation induction performing operation (S170) of, under condition B, when the physical condition enters another standard range set by the user, asking the user whether to start meditation, and when an operation button input or a touch button input is performed, performing meditation according to a preset program; and
a meditation end determination operation (S180) of, when a time to complete a meditation process according to the preset program is imminent, asking the user whether to end the meditation and whether to continue the meditation.
